SEQUENCE LISTING (3 sequences)

SEQ ID NO: 1 - DNA, 1731 bp, Homo sapiens
ggatcctgcagcccgagtcccgtcctcaggcttccccaatccaggggactcggcgccgggacgctgctatggacgacattttcactcagtgccgggagggcaacgcagtcgccgttcgcctgtggctggacaacacggagaacgacctcaaccagggggacgatcatggcttctcccccttgcactgggcctgccgagagggccgctctgctgtggttgagatgttgatcatgcggggggcacggatcaatgtaatgaaccgtggggatgacacccccctgcatctggcagccagtcatggacaccgtgatattgtacagaagctattgcagtacaaggcagacatcaatgcagtgaatgaacatgggaatgtgcccctgcactatgcctgtttttggggccaagatcaagtggcagaggacctggtggcaaatggggcccttgtcagcatctgtaacaagtatggagagatgcctgtggacaaagccaaggcacccctgagagagcttctccgagagcgggcagagaagatgggccagaatctcaaccgtattccatacaaggacacattctggaaggggaccacccgcactcggccccgaaatggaaccctgaacaaacactctggcattgacttcaaacagcttaacttcctgacgaagctcaacgagaatcactctggagagctatggaagggccgctggcagggcaatgacattgtcgtgaaggtgctgaaggttcgagactggagtacaaggaagagcagggacttcaatgaagagtgtccccggctcaggattttctcgcatccaaatgtgctcccagtgctaggtgcctgccagtctccacctgctcctcatcctactctcatcacacactggatgccatatggatccctctacaatgtactacatgaaggcaccaatttcgtcgtggaccagagccaggctgtgaagtttgctttggacatggcaaggggcatggccttcctacacacactagagcccctcatcccacgacatgcactcaatagccgtagtgtaatgattgatgaggacatgactgcccgaattagcatggctgatgtcaagttctctttccaatgtcctggtcgcatgtatgcacctgcctgggtagcccccgaagctctgcagaagaagcctgaagacacaaacagacgctcagcagacatgtggagttttgcagtgcttctgtgggaactggtgacacgggaggtaccctttgctgacctctccaatatggagattggaatgaaggtggcattggaaggccttcggcctaccatcccaccaggtatttcccctcatgtgtgtaagctcatgaagatctgcatgaatgaagaccctgcaaagcgacccaaatttgacatgattgtgcctatccttgagaagatgcaggacaagtaggactggaaggtccttgcctgaactccagaggtgtcgggacatggttgggggaatgcacctccccaaagcagcaggcctctggttgcctcccccgcctccagtcatggtactaccccagccatggggtccatccccttcccccatccctaccactgtggccccaagaggggcgggctcagagctttgtcacttgccacatggtgtctcccaacatgggagggatcagccccgcctgtcacaataaagtttattatgaaaaaaaaaaaaaaaaaaaaaaaaaaaaaaaaaaaaaaaaaaaaaaaaa

SEQ ID NO: 2 - DNA, 4091 bp, Homo sapiens
gaaagccgcagcctcagtcccgccgccgcccgctgcgtccgcccagcgccagctccgcgtcccgaccggcccgcggcagcctgcgccgcgccatggccacctccccgcagaagtcgccttctgtccccaagtctcccactcccaagtcgcccccgtcccgcaagaaagatgattccttcttggggaaactcggagggaccctggcccggaggaagaaagccaaggaggtgtccgagctgcaggaggagggaatgaacgccatcaacctgcccctcagcccaattccctttgagctggaccccgaggacacgatgctggaggagaatgaggtgcgaacaatggtggatccaaactcacgcagtgaccccaagcttcaagaactgatgaaggtattaattgactggattaatgatgtgttggttggagaaagaatcattgtgaaagacctagctgaagatttgtatgatggacaagtcctgcagaagcttttcgagaaactggagagtgagaagctaaatgtggctgaggtcacccagtcagagattgctcagaagcaaaaactgcagactgtcctggagaagatcaatgaaaccctgaaacttcctcccaggagcatcaagtggaatgtggattctgttcatgccaagagcctggtggccatcttacacctgctcgttgctctgtctcagtatttccgcgcaccaattcgactcccagaccatgtttccatccaagtggttgtggtccagaaacgagaaggaatcctccagtctcggcaaatccaagaggaaataactggtaacacagaggctctttccgggaggcatgaacgtgatgcctttgacaccttgttcgaccatgccccagacaagctgaatgtggtgaaaaagacactcatcactttcgtgaacaagcacctgaataaactgaacctggaggtcacagaactggaaacccagtttgcagatggggtgtacctggtgctgctcatggggctcctggagggctactttgtgcccctgcacagcttcttcctgaccccggacagctttgaacagaaggtcttgaatgtctcctttgcctttgagctcatgcaagatggagggttggaaaagccaaaaccgcggccagaagacatagtcaactgtgacctgaaatctacactacgagtgttgtacaacctcttcaccaagtaccgtaacgtggagtgaggggctgccctgggcccaccactgcccaagagttcttgctgttggcgtactggaccctcctccgaactgccttaccctgcttattcctgtctcttgcactgtgctctcccacaagtccagctgcaacccagagatagtggaaactgaaattaggaaggaaatcatcaataactcagtgggctgacccatccctcccaggcgctggggaccaacctagcaatgaaggttgggaaggttgttcccttcccggtgccaggtccagatttccctccatgatttgggaaccagcttaggcaaaagagtccccacaagatgaaaataaagatcctagttaccattcaaaggatgctaactgtgtgtcaggccccacactaagtgctctgctctgatatactcaaggccattaatcttcaggactcccattgacgtaggtgtttcattccccttttacagatgaggaaactaaggcttggaggttaaatgacttgccagaagttggaatttttttcctctttgaacataacctctcccttctccctaaaggtaaccactattctgagtccaatcatcaaggttttgcttttctttttagctaagtatgcattcctcaatagtagacagtacaacatgtttataacaagccaattacattatgttctttgcatgttctaaagttgtgtatgtgtgtgcacatctgagcacgtgcacatgtacacctgagccaaaaacacgagaacccactgatctcaccactggggcaagctaggtcagagcttagtgattcacactgaaattggcaaattggatttaacccaattaatagtgtgtgtgtggcaggagtcatgtccctcacatcctttgtacaaatgaaaattactcttaattccttcagatttataataactctgtactttggtttcagggtgacatttgggaaggattttgtttagaattaatggagtggcacattttgcagcctttttgcttgattgcatgtaatggaaatgccctatattttcctgcaaaataagtactaaattcattatcgttaagcaaatgtacaatatgctcaggcaccgcagagagctgggcacgggcccatgtgagcatcactttggaagtagggctcttcaacagggacccttgaactttaaagaaaggaacttctttttgccttctaattgatcatttagactattctggctaagtctgcccacatgtaattaccggctaattcaagcgaggaaaaatgtaagtcatttagaccaaagccaagcagtttctttgcgtgggttactcaagggcttgtggttacttgtatctcctctatgtgaacttgactttgaaagacagagctctagtgtgccagcctgctaagtcctgtaagaatagggaagggcggaggggggtgggcagtgactaggggacgagaagcatggggaaaatatttgcactctaaacatacagagatagaggtggggcttgtggtacttaacacttgtagccatcaactgactgagaccttgggctaaataatcaattgtgctgatattacatctcgttatggaatgttcctaaatatgccaggtagacaccagcccaagtaccctcctccagaagtctgtgactaccttgtcactactttaggcccattccacaaagcccatctctggtttgagaattcattttgatctgtatctacaccacccaaagttaggcctcctataatgtccaaaacattcctttcagcctttttatttcttactgtactgtctcttactgtactgtctatctgcagtaattgaggacccataaaatttagataactacatgtctttgctcttagaattgtcactcagcataatgagcatttaacatacaaaggcaatgtactgttttgtgttgatctatgtaaaagaatacaattcttttttacataattagtgaaattttattttttattaggaaacactaaatagtgtaatatttcttttgcttttaaaaaaattcctggtagcaaatcaagataaataattgcttcattttcttgagcaatactgaagcaggatgaagtaagaggaatgcattcatttaaacatgctttgctttatgaattttgtctcttttttggtctctttttcttatattcaagttacaaatgtacaagtatccttactaagagtgctccttttgtattttacatatatacagtatgaaaatacattggaacactaggaaagtttttaaataacagttctaatttatcagaaaattgtgttttgggattgagttctttgtctcagcccagaatcccaggtcctgggcctggttttctaatgctgtcatctcagttcgatattttactttagaatctggaatctcctacttaatatatgaccatgactttgaaaggcaaaagaggaatcaagaataaataaaacaaacttaatcttcatctttaaaaaaaagaaaaaagaaaccaaaatgagaatcaacacttcataggctcactgggttttcttttcttttctgttaatttaaactcagttatttttaatgcttaatacatacatggtgcaaaatttaaaaagcgcaaataggtatctagtggaaaacctaagcctccctctctctccggcacccattacctctccctggaggcaactgttttgatccatttcttacacacactgccagagatactctaggcatgtaaagcacaaacatacatataaaatctgcgggcttcaaaaaatataagtaggatgtcatctatactgtcatacactttgttttttatcacttacttaatgttatatcttggatattgtattaccctgggtattaaaaagaactcctttcacattttaaaataaaaaa

SEQ ID NO: 3 - DNA, 1877 bp, Homo sapiens
acaaagactgtgtcactactttgtggaatgttattttagaacttccagaatgcctcagtaaatcctgactgccatgtaactgtgtactgtgctgctgagctcttaacatgaagcagcagcagtgaccctggcgggccaggctgacgtgcgtgggaacagtgggagtgaccctggctgggaagcagggaggcctggatattgtttctcctggctctgtgtcctgccattctcatccctgtgctcagagctctcaagcacccacgatggccttctcaggccgagcgcgcccctgcattatcccagagaacgaagaaatcccccgagcagcccttaacactgtccacgaggccaatgggaccgaggacgagagggctgtttccaaactgcagcgcaggcacagtgacgtgaaagtctacaaggagttctgtgacttttatgcgaaattcaacatggccaacgccctggccagcgccacttgcgagcgctgcaagggcggctttgcgcccgctgagaagatcgtgaacagtaatggggagctgtaccatgagcagtgtttcgtgtgcgctcagtgcttccagcagttcccagaaggactcttctatgagtttgaaggaagaaagtactgtgaacatgactttcagatgctctttgccccttgctgtcatcagtgtggtgaattcatcattggccgagttatcaaagccatgaataacagctggcatccggagtgcttccgctgtgacctctgccaggaagttctggcagatatcgggtttgtcaagaatgctgggagacacctgtgtcgcccctgtcataatcgtgagaaagccagaggccttgggaaatacatctgccagaaatgccatgctatcatcgatgagcagcctctgatattcaagaacgacccctaccatccagaccatttcaactgcgccaactgcgggaaggagctgactgccgatgcacgggagctgaaaggggagctatactgcctcccatgccatgataaaatgggggtccccatctgtggtgcttgccgacggcccatcgaagggcgcgtggtgaacgctatgggcaagcagtggcatgtggagcattttgtttgtgccaagtgtgagaaaccctttcttggacatcgccattatgagaggaaaggcctggcatattgtgaaactcactataaccagctatttggtgatgtttgcttccactgcaatcgtgttatagaaggtgatgtggtctctgctcttaataaggcctggtgcgtgaactgctttgcctgttctacctgcaacactaaattaacactcaagaataagtttgtggagtttgacatgaagccagtctgtaagaagtgctatgagaaatttccattggagctgaagaaaagacttaagaaactagctgagaccttaggaaggaaataagttcctttattttttcttttctatgcaagataagagattaccaacattacttgtcttgatctacccatatttaaagctatatctcaaagcagttgagagaagaggacctatatgaatggttttatgtcatttttttaattaaaaaagaaaaattcatataatcgtgtttaaaacacaaatgaagtcagtatttgcctttgttaacccttatccatttgttgacatgtagactgtttacaaaaaaaaaacacatggttaaatgttaaattttaattaaggcccccaaaaattaaatataactttttaaaatgaaaggagtcaccttttacatgactcaggtgaaaaaacagtataaacattaatttactttgtgttcaaaagaaaattccaactgctgttggggaaggacacagaaaagaaaaataaccacccaagaaaaacaaaaaaaaa

(19) United States
(12) Patent Application Publication
(10) Pub. No.: US 2012/0095029 A1
(43) Pub. Date: Apr. 19, 2012

(54) IPP COMPLEX AS MARKER FOR ERLOTINIB TREATMENT

(75) Inventors: Angélique Augustin, Flueh (CH); Guillemette Duchateau-Nguyen, France (FR); Laurent Essioux, Attenschwiller (FR); Sabrina Golling, Oltingue (FR); Barbara Klughammer, Rheinfelden (DE); Jens Lamerz, Offenburg (DE); Hanno Langen, Loerrach (DE); Héléne Meistermann, Riedisheim (FR); Stefan Scheiblich, Penzberg (DE); Manuel Tzouros, Biel/Bienne (CH)

(73) Assignee: Hoffmann-La Roche Inc., Nutley, NJ (US)

(21) Appl. No.: 13/270,530

(22) Filed: Oct. 11, 2011

(30) Foreign Application Priority Data
Oct. 15, 2010 (EP) 101876837
Jul. 13, 2011 (EP) 111738357

(51) Int. Cl. A61K 31/517; A61P 35/00; G01N 33/566; C40B 30/04

(52) U.S. Cl. 514/266.4; 506/9; 435/7.23

(57) ABSTRACT

The present invention provides biomarkers which are predictive for the clinical benefit of erlotinib hydrochloride treatment in cancer patients.

This application claims priority under 35 USC §119(a) to European patent application no. 10187683.7, filed on Oct. 15, 2010 and European patent application no. 11173835.7, filed on Jul. 13, 2011, the entirety of which are incorporated herein by reference.

The present invention provides biomarkers that are predictive for the clinical benefit of erlotinib hydrochloride treatment in cancer patients.

A number of human malignancies are associated with aberrant or over-expression of the epidermal growth factor receptor (EGFR). EGF, transforming growth factor-α (TGF-α), and a number of other ligands bind to the EGFR, stimulating autophosphorylation of the intracellular tyrosine kinase domain of the receptor. A variety of intracellular pathways are subsequently activated, and these downstream events result in tumor cell proliferation in vitro. It has been postulated that stimulation of tumor cells via the EGFR may be important for both tumor growth and tumor survival in vivo.

Early clinical data with Tarceva®, an inhibitor of the EGFR tyrosine kinase, indicate that the compound is safe and generally well tolerated at doses that provide the targeted effective concentration (as determined by preclinical data). Clinical phase I and II trials in patients with advanced disease have demonstrated that Tarceva® has promising clinical activity in a range of epithelial tumors. Indeed, Tarceva® has been shown to be capable of inducing durable partial remissions in previously treated patients with head and neck cancer, and NSCLC (Non small cell lung cancer) of a similar order to established second line chemotherapy, but with the added benefit of a better safety profile than chemo therapy and improved convenience (tablet instead of intravenous [i.v.] administration). A randomised, double-blind, placebo-controlled trial (BR.21) has shown that single agent Tarceva® significantly prolongs and improves the survival of NSCLC patients for whom standard therapy for advanced disease has failed.

Tarceva® (erlotinib hydrochloride) is a small chemical molecule; it is an orally active, potent, inhibitor of the EGFR tyrosine kinase (EGFR-TKI).

Iressa™ (gefitinib) is a small chemical molecule; it is an orally active inhibitor of the EGFR tyrosine kinase (EGFR-TKI).

Lung cancer is the major cause of cancer-related death in North America and Europe. In the United States, the number of deaths secondary to lung cancer exceeds the combined total deaths from the second (colon), third (breast), and fourth (prostate) leading causes of cancer deaths combined. About 75% to 80% of all lung cancers are NSCLC, with approximately 40% of patients presenting with locally advanced and/or unresectable disease. This group typically includes those with bulky stage IIIA and IIIB disease, excluding malignant pleural effusions.

The crude incidence of lung cancer in the European Union is 52.5, the death rate 48.7 cases/100000/year. Among men the rates are 79.3 and 78.3, among women 21.6 and 20.5, respectively. NSCLC accounts for 80% of all lung cancer cases. About 90% of lung cancer mortality among men, and 80% among women, is attributable to smoking.

In the US, according to the American Cancer Society, during 2004, there were approximately 173,800 new cases of lung cancer (93,100 in men and 80,700 in women) and were accounting for about 13% of all new cancers. Most patients die as a consequence of their disease within two years of diagnosis. For many NSCLC patients, successful treatment remains elusive. Advanced tumors often are not amenable to surgery and may also be resistant to tolerable doses of radiotherapy and chemotherapy. In randomized trials the currently most active combination chemotherapies achieved response rates of approximately 30% to 40% and a 1-year survival rate between 35% and 40%. This is really an advance over the 10% 1-year survival rate seen with supportive care alone (Shepherd 1999).

Until recently therapeutic options for relapsed patients following relapse were limited to best supportive care or palliation. A recent trial comparing docetaxel (Taxotere) with best supportive care showed that patients with NSCLC could benefit from second line chemotherapy after cisplatin-based first-line regimens had failed. Patients of all ages and with ECOG performance status of 0, 1, or 2 demonstrated improved survival with docetaxel, as did those who had been refractory to prior platinum-based treatment. Patients who did not benefit from therapy included those with weight loss of 10%, high lactate dehydrogenase levels, multi-organ involvement, or liver involvement. Additionally, the benefit of docetaxel monotherapy did not extend beyond the second line setting. Patients receiving docetaxel as third-line treatment or beyond showed no prolongation of survival. Single-agent docetaxel became a standard second-line therapy for NSCLC. Recently another randomized phase III trial in second line therapy of NSCLC compared pemetrexed (Alimta®) with docetaxel. Treatment with pemetrexed resulted in a clinically equivalent efficacy but with significantly fewer side effects compared with docetaxel.

It has long been acknowledged that there is a need to develop methods of individualizing cancer treatment. With the development of targeted cancer treatments, there is a particular interest in methodologies which could provide a molecular profile of the tumor target, (i.e. those that are predictive for clinical benefit). Proof of principle for gene expression profiling in cancer has already been established with the molecular classification of tumor types which are not apparent on the basis of current morphological and immunohistochemical tests. Two separate disease entities were differentiated with differing prognoses from the single current classification of diffuse large B-cell lymphoma using gene expression profiling.

It has been shown that NSCLC patients carrying an activating EGFR mutation in their tumor respond especially well to treatment with tyrosine kinase inhibitors like erlotinib and gefitinib (Paz-Ares et al. 2010). Whereas gefitinib does not seem to confer clinical benefit to an unselected patient population (Thatcher et al. Lancet 2005) erlotinib does (Shepherd et al. NEJM 2005; Cappuzzo et al 2010). Therefore, it is an aim of the present invention to provide expression biomarkers that are predictive for the clinical benefit of erlotinib hydrochloride treatment in cancer patients and are non-predictive for the clinical benefit of gefitinib treatment in cancer patients.

The ECM (Extracellular Matrix) provides the structural framework for the formation of tissues and organs. The ECM binds to substrate adhesion molecules on the surface of cells and influences various intracellular signaling pathways that regulate survival, proliferation, polarity and differentiation. The important families of adhesion molecules that bind to the ECM are the Integrins. Integrins consist of Alpha and Beta-subunits and are composed of large extracellular domains and relatively small cytoplasmic domains. Ligand binding activates signaling cascades that lead to the assembly of a multiprotein complex at the site of cell adhesion to the ECM. These events have two important impacts on the cell: they forge a connection between the ECM and the Actin cytoskeleton, and they alter the fluxes of many intracellular signaling pathways. Three proteins which act as important regulators of Integrin mediated signaling are the ILK (Integrin Linked Kinase) and the adaptor proteins PINCH (Particularly Interesting Cys-His-rich Protein) and Pary (Parvin), among which ILK is the major regulator of Integrin mediated signaling. These molecules form a heterotrimeric complex, which is referred to as the IPP complex. The main function of ILK is to connect Integrins to the cytoskeleton. ILK binds PINCH through its N-terminal domain and binds alpha-Parvin through its C-terminal domain, resulting in formation of PINCH-ILK-alpha-Parvin ternary complexes (IPP complex). The IPP complex functions both as an adaptor between Integrins and the Actin cytoskeleton and as a hub that regulates several signaling pathways.

Wild-type EGFR containing human NSCLC lines show greater sensitivity to erlotinib hydrochloride before EMT (Thomson et al. (2005)). Epithelial-mesenchymal transition (EMT) is a phenotypic conversion of biological cells characterized by loss of cell adhesion, repression of E-cadherin expression, and increased cell mobility. Several oncogenic pathways (e.g. peptide growth factors, integrin, etc.) are believed to induce EMT. EMT is a characteristic feature of cells undergoing proliferation, it has been suggested to play a significant role in tumor progression and metastases. Increased expression of integrin-linked kinase is associated with shorter survival in non-small cell lung cancer (Takanami et al. (2005)).

The MERIT study is a non-randomized, open-label efficacy marker identification trial for Tarceva in second-line non-small cell lung cancer patients.

The term “biomarker” means a substance used as an indicator of a biological state, i.e. of biological processes or pharmacologic response to a therapeutic interaction. A biomarker indicates a change in expression or state of a protein that correlates with the risk or progression of a disease, or with the susceptibility of the disease to a given treatment.

The term “marker gene” means a gene used to determine if a piece of DNA has been successfully inserted into the host organism.

Gene expression is the process in which information from a gene is used in the synthesis of a functional gene product, e.g. a protein. The term “expression level” means the level at which a particular gene is expressed within a cell, tissue or organism. The term “expression level” in context with proteins reflects the amount of a particular protein present in a cell at a certain time.

“Chemical proteomics” is a mass-spectrometry based affinity chromatography approach that uses immobilized small molecules as bait to capture and identify interacting protein complexes from an entire proteome. It can be applied on kinase inhibitors for instance, to explore their mode of action, or on natural products for target identification.

The term “activation level” reflects the potential of a particular protein to be active in a cell at a certain time. An example are kinases being phosphorylated at a certain site to be able to modify their substrate.

The term “a value representative of an expression level of the IPP complex in tumors of a population of patients deriving no clinical benefit from the treatment” refers to an estimate of a mean expression level of the marker gene in tumors of a population of patients who do not derive a clinical benefit from the treatment. Clinical benefit was defined as either having an objective response or disease stabilization for ≧12 weeks.

In a particular embodiment, the expression level of the marker gene is determined by microarray technology or other technologies that assess RNA expression levels like quantitative RT-PCR, or by any method looking at the expression level of the respective protein, eg immunohistochemistry (IHC). The construction and use of gene chips are well known in the art. see, U.S. Pat. Nos. 5,202,231; 5,445,934; 5,525,464; 5,695,940; 5,744,305; 5,795,716 and 1 U.S. Pat. No. 5,800,992. See also, Johnston, M. Curr. Biol. 8:R171-174 (1998); Iyer VR et al., Science 283:83-87 (1999). Of course, the gene expression level can be determined by other methods that are known to a person skilled in the art such as e.g. northern blots, RT-PCR, real time quantitative PCR, primer extension, RNase protection, RNA expression profiling.

The marker gene of the present invention can be combined with other biomarkers to biomarker sets, in particular with EGFR Biomarkers. Biomarker sets can be built from any combination of predictive biomarkers to make predictions about the effect of erlotinib hydrochloride treatment in cancer patients. The biomarkers and biomarkers sets described herein can be used, for example, to predict how patients with cancer will respond to therapeutic intervention with erlotinib hydrochloride.

The term “gene” as used herein comprises variants of the gene. The term “variant” relates to nucleic acid sequences which are substantially similar to the nucleic acid sequences given by the GenBank accession number. The term “substantially similar” is well understood by a person skilled in the art. In particular, a gene variant may be an allele which shows nucleotide exchanges compared to the nucleic acid sequence of the most prevalent allele in the human population. In an embodiment, such a substantially similar nucleic acid sequence has a sequence similarity to the most prevalent allele of at least 80%. In an embodiment, such a substantially similar nucleic acid sequence has a sequence similarity to the most prevalent allele of at least 85%. In an embodiment, such a substantially similar nucleic acid sequence has a sequence similarity to the most prevalent allele of at least 90%. In an embodiment, such a substantially similar nucleic acid sequence has a sequence similarity to the most prevalent allele of at least 95%. The term “variants” is also meant to relate to splice variants.

Techniques for the detection and quantification of gene expression of the genes described by this invention include, but are not limited to northern blots, RT-PCR, real time quantitative PCR, primer extension, RNase protection, RNA expression profiling and related techniques. These techniques are well known to those of skill in the art see e.g. Sambrook J et al., Molecular Cloning: A Laboratory Manual, Third Edition (Cold Spring Harbor Press, Cold Spring Harbor, 2000).

Techniques for the detection of protein expression of the respective genes described by this invention include, but are not limited to immunohistochemistry (IHC).

The biomarkers of the present invention are an individualized therapy for patients with cancer, in particular patients with refractory NSCLC. This individualized therapy will allow treating physicians to select the most appropriate agent out of the existing drugs for cancer therapy, in particular NSCLC. The benefit of individualized therapy for each future patient are: response rates/number of benefiting patients will increase and the risk of adverse side effects due to ineffective treatment will be reduced.

It is to be understood that the terminology employed herein is for the purpose of describing particular embodiments, and is not intended to be limiting. Further, although any methods, devices and materials similar or equivalent to those described herein can be used in the practice or testing of the invention, embodiments of the methods, devices and materials are now described.

The present invention surprisingly found that the response of a cancer patient to treatment with erlotinib hydrochloride can be predicted by determining the activation levels of ILK, alpha-parvin and PINCH proteins together, individually or each individually combined in a tumor sample of a patient. Further, the difference in clinical outcomes can be attributed to differences in the protein interaction profiles of erlotinib and gefitinib, determined by chemical proteomics.

In a certain object the present invention relates to an in vitro method of predicting the response of a cancer patient to treatment with erlotinib hydrochloride comprising:

determining the expression levels of one, two or three genes selected from the group consisting of ILK, alpha-parvin and PINCH genes in a tumor sample of a patient and comparing the expression levels of the ILK, alpha-parvin and PINCH genes together or individually to a value representative of the expression levels of ILK, alpha-parvin and PINCH genes together or individually in tumors of a population of patients deriving no clinical benefit from the treatment, wherein a lower expression level of ILK, alpha-parvin and PINCH genes together or individually in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the expression levels of the ILK, alpha-parvin and PINCH genes in a tumor sample of a patient and comparing the expression levels of the ILK, alpha-parvin and PINCH genes together to a value representative of the expression levels of ILK, alpha-parvin and PINCH genes together in tumors of a population of patients deriving no clinical benefit from the treatment, wherein a lower expression level of ILK, alpha-parvin and PINCH genes together in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the expression levels of the ILK and PINCH genes in a tumor sample of a patient and comparing the expression levels of the ILK and PINCH genes together to a value representative of the expression levels of ILK and PINCH genes together in tumors of a population of patients deriving no clinical benefit from the treatment, wherein a lower expression level of ILK and PINCH genes together in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the expression levels of the ILK and alpha-parvin genes in a tumor sample of a patient and comparing the expression levels of the ILK and alpha-parvin genes together to a value representative of the expression levels of ILK and alpha-parvin genes together in tumors of a population of patients deriving no clinical benefit from the treatment, wherein a lower expression level of ILK and alpha-parvin genes together in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the expression levels of the alpha-parvin and PINCH genes in a tumor sample of a patient and comparing the expression levels of the alpha-parvin and PINCH genes together to a value representative of the expression levels of alpha-parvin and PINCH genes together in tumors of a population of patients deriving no clinical benefit from the treatment, wherein a lower expression level of alpha-parvin and PINCH genes together in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the expression levels of the ILK gene in a tumor sample of a patient and comparing the expression level of the ILK gene to a value representative of the expression level of ILK gene in tumors of a population of patients deriving no clinical benefit from the treatment, wherein a lower expression level of ILK gene in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the expression levels of the PINCH gene in a tumor sample of a patient and comparing the expression level of the PINCH gene to a value representative of the expression level of PINCH gene in tumors of a population of patients deriving no clinical benefit from the treatment, wherein a lower expression level of PINCH gene in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the expression levels of the alpha-parvin gene in a tumor sample of a patient and comparing the expression level of the alpha-parvin gene to a value representative of the expression level of alpha-parvin gene in tumors of a population of patients deriving no clinical benefit from the treatment, wherein a lower expression level of alpha-parvin gene in the tumor sample of the patient is indicative for a patient who will derive clinical benefit from the treatment.

In a certain object the present invention relates to a method as described herein, wherein the expression level is determined by microarray technology.

In a certain object the present invention relates to an in vitro method of predicting the response of a cancer patient to treatment with erlotinib hydrochloride comprising:

determining the activation level of one, two or three proteins selected from the group consisting of ILK, alpha-parvin and PINCH proteins, in a tumor sample of a patient and comparing the activation levels of the ILK, alpha-parvin and PINCH proteins together or individually to a value representative of the activation levels of ILK, alpha-parvin and PINCH proteins together or individually in tumors of a population of patients deriving no clinical benefit from the treatment, wherein a lower activation level of ILK, alpha-parvin and PINCH proteins together or individually in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the activation level of the ILK, alpha-parvin and PINCH proteins, in a tumor sample of a patient and comparing the activation levels of the ILK, alpha-parvin and PINCH proteins together to a value representative of the activation levels of ILK, alpha-parvin and PINCH proteins together in tumors of a population of patients deriving no clinical benefit from the treatment, wherein a lower activation level of ILK, alpha-parvin and PINCH proteins together in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the activation level of the ILK protein in a tumor sample of a patient and comparing the activation level of the ILK protein to a value representative of the activation level of ILK protein in tumors of a population of patients deriving no clinical benefit from the treatment, wherein a lower activation level of ILK protein in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the activation level of the PINCH protein in a tumor sample of a patient and comparing the activation level of the PINCH protein to a value representative of the activation level of PINCH protein in tumors of a population of patients deriving no clinical benefit from the treatment, wherein a lower activation level of PINCH protein in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the activation level of the ILK and PINCH proteins, in a tumor sample of a patient and comparing the activation levels of the ILK and PINCH proteins together to a value representative of the activation levels of ILK and PINCH proteins together in tumors of a population of patients deriving no clinical benefit from the treatment, wherein a lower activation level of ILK and PINCH proteins together in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the activation level of the alpha-parvin and PINCH proteins, in a tumor sample of a patient and comparing the activation levels of the alpha-parvin and PINCH proteins together to a value representative of the activation levels of alpha-parvin and PINCH proteins together in tumors of a population of patients deriving no clinical benefit from the treatment, wherein a lower activation level of alpha-parvin and PINCH proteins together in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the activation level of the ILK and alpha-parvin proteins, in a tumor sample of a patient and comparing the activation levels of the ILK and alpha-parvin proteins together to a value representative of the activation levels of ILK and alpha-parvin proteins together in tumors of a population of patients deriving no clinical benefit from the treatment, wherein a lower activation level of ILK and alpha-parvin proteins together in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the activation level of the alpha-parvin protein in a tumor sample of a patient and comparing the activation level of the alpha-parvin protein to a value representative of the activation level of alpha-parvin protein in tumors of a population of patients deriving no clinical benefit from the treatment, wherein a lower activation level of alpha-parvin protein in the tumor sample of the patient is indicative for a patient who will derive clinical benefit from the treatment.

In a certain object the present invention relates to an in vitro method of predicting the response of a cancer patient to treatment with erlotinib hydrochloride comprising:

determining the phosphorylation levels of ILK protein in a tumor sample of a patient and comparing the phosphorylation levels of the ILK to a value representative of the phosphorylation levels of ILK in tumors of a population of patients deriving no clinical benefit from the treatment, wherein a different phosphorylation level of ILK protein in the tumor sample of the patient is indicative for a patient who will derive clinical benefit from the treatment.

In a certain object the present invention relates to a method for selection of cancer treatment for a human patient, the method comprising:
i) determining the expression levels of one, two or three genes selected from the group consisting of ILK, alpha-parvin and PINCH genes in a tumor sample of a patient,
ii) comparing the expression levels of one, two or three genes selected from the group consisting of ILK, alpha-parvin and PINCH genes to a value representative of the expression levels of one, two or three genes selected from the group consisting of ILK, alpha-parvin and PINCH genes in tumors of a population of patients deriving no clinical benefit from the treatment,
iii) wherein a lower expression levels of one, two or three genes selected from the group consisting of ILK, alpha-parvin and PINCH genes in the tumor sample of the patient is indicative for a patient who will derive clinical benefit from treatment with erlotinib hydrochloride, and
iv) selecting erlotinib hydrochloride treatment as the cancer treatment for the patient.

In a certain object the present invention relates to a method of treating cancer in a patient comprising administering an effective amount of erlotinib hydrochloride to the patient, provided that the expression levels of one, two or three genes selected from the group consisting of ILK, alpha-parvin and PINCH genes in a tumor sample of a patient are lower than the expression levels of expression levels of one, two or three genes selected from the group consisting of ILK, alpha-parvin and PINCH genes in the tumors of a population of patients deriving no clinical benefit from treatment with erlotinib hydrochloride.

In a certain object the present invention relates to a method for selection of cancer treatment for a human patient, the method comprising:
i) determining the activation levels of one, two or three proteins selected from the group consisting of ILK, alpha-parvin and PINCH proteins in a tumor sample of a patient,
ii) comparing the activation levels of one, two or three proteins selected from the group consisting of ILK, alpha-parvin and PINCH proteins to a value representative of the activation levels of one, two or three proteins selected from the group consisting of ILK, alpha-parvin and PINCH proteins in tumors of a population of patients deriving no clinical benefit from the treatment,
iii) wherein a lower activation level of one, two or three proteins selected from the group consisting of ILK, alpha-parvin and PINCH proteins in the tumor sample of the patient is indicative for a patient who will derive clinical benefit from treatment with erlotinib hydrochloride, and
iv) selecting erlotinib hydrochloride treatment as the cancer treatment for the patient.

In a certain object the present invention relates to a method of treating cancer in a patient comprising administering an effective amount of erlotinib hydrochloride to the patient, provided that the activation levels of one, two or three proteins selected from the group consisting of ILK, alpha-parvin and PINCH proteins in a tumor sample of a patient are lower than the activation levels of expression levels of one, two or three proteins selected from the group consisting of ILK, alpha-parvin and PINCH proteins in the tumors of a population of patients deriving no clinical benefit from treatment with erlotinib hydrochloride.

In a certain object the present invention relates to a method as described herein, concerning ILK, alpha-parvin and PINCH together.

In a certain object the present invention relates to a method as described herein, concerning ILK and PINCH together.

In a certain object the present invention relates to a method as described herein, concerning alpha-parvin and PINCH together.

In a certain object the present invention relates to a method as described herein, concerning ILK and alpha-parvin together.

In a certain object the present invention relates to a method as described herein, concerning ILK.

In a certain object the present invention relates to a method as described herein, concerning alpha-parvin.

In a certain object the present invention relates to a method as described herein, concerning PINCH.

In a certain object the present invention relates to a method as described herein, wherein the cancer is non-small cell lung cancer (NSCLC).

In a certain object the present invention relates to a method as described herein, wherein a cancer patient does not respond to treatment with gefitinib.

In a certain object the present invention relates to an use of one, two or three genes selected from the group consisting of ILK, alpha-parvin and PINCH genes for predicting the response of a cancer patient to erlotinib hydrochloride treatment.

In a certain object the present invention relates to an use of ILK, alpha-parvin and PINCH genes for predicting the response of a cancer patient to erlotinib hydrochloride treatment.

In a certain object the present invention relates to an use of alpha-parvin and PINCH genes for predicting the response of a cancer patient to erlotinib hydrochloride treatment.

In a certain object the present invention relates to an use of ILK and PINCH genes for predicting the response of a cancer patient to erlotinib hydrochloride treatment.

In a certain object the present invention relates to an use of ILK and alpha-parvin genes for predicting the response of a cancer patient to erlotinib hydrochloride treatment.

In a certain object the present invention relates to an use of erlotinib hydrochloride for treating cancer, wherein

i) the activation levels of one, two or three proteins selected from the group consisting of ILK, alpha-parvin and PINCH proteins in a tumor sample of a patient are measured,

ii) the expression levels of the ILK, alpha-parvin and PINCH genes together or individually are compared to a value representative of the expression levels of ILK, alpha-parvin and PINCH genes together or individually in tumors of a population of patients deriving no clinical benefit from the treatment, and

iii) erlotinib hydrochloride is administered to a patient having a lower expression level of ILK, alpha-parvin and PINCH genes together or individually.

In a certain object the present invention relates to an use of erlotinib hydrochloride for treating cancer, wherein the activation levels of ILK, alpha-parvin and PINCH proteins together are concerned.

In a certain object the present invention relates to an use of erlotinib hydrochloride for treating cancer, wherein the activation level of ILK protein is concerned.

In a certain object the present invention relates to an use of erlotinib hydrochloride for treating cancer, wherein the activation level of alpha-parvin protein is concerned.

In a certain object the present invention relates to an use of erlotinib hydrochloride for treating cancer, wherein the activation level of PINCH protein is concerned.

In a certain object the present invention relates to an use of erlotinib hydrochloride for treating cancer, wherein the activation levels of ILK and alpha-parvin proteins are concerned.

In a certain object the present invention relates to an use of erlotinib hydrochloride for treating cancer, wherein the activation levels of ILK and PINCH proteins are concerned.

In a certain object the present invention relates to an use of erlotinib hydrochloride for treating cancer, wherein the activation levels of alpha-parvin and PINCH proteins are concerned.

In a certain object the present invention relates to an use as described herein, concerning ILK.

In a certain object the present invention relates to an use as described herein, concerning alpha-parvin.

In a certain object the present invention relates to an use as described herein, concerning PINCH.

In a certain object the present invention relates to an use as described herein, wherein the cancer is NSCLC.

In a certain object the present invention relates to an use as described herein, wherein a cancer patient does not respond to treatment with gefitinib.

In a certain object the present invention relates to a kit for detecting expression levels of one, two or three genes selected from the group consisting of ILK, alpha-parvin and PINCH genes, the kit comprising i) a compound capable of specifically detecting expression levels of one, two or three genes selected from the group consisting of ILK, alpha-parvin and PINCH genes, and ii) instructions for using said kit to predict responsiveness of a patient suffering from cancer treatment with erlotinib hydrochloride, wherein decreased expression levels of one, two or three genes selected from the group consisting of ILK, alpha-parvin and PINCH genes as compared to a reference sample indicates that a patient may benefit from treatment with erlotinib hydrochloride.

In a certain object the present invention relates to a kit as described herein, concerning ILK.

In a certain object the present invention relates to a kit as described herein, concerning alpha-parvin.

In a certain object the present invention relates to a kit as described herein, concerning PINCH.

In a certain object the present invention relates to a kit as described herein, wherein the cancer is NSCLC.

In a certain object the present invention relates to a kit as described herein, wherein a cancer patient does not respond to treatment with gefitinib.

In a further object the present invention provides a therapeutic method of treating a cancer patient identified by the in vitro method of the present invention. Said therapeutic method comprises administering erlotinib hydrochloride to the patient who has been selected for treatment based on the predictive expression and/or activation pattern of the genes of enclosed SEQ IDs 1, 2 and 3. A particular cancer to be treated is NSCLC.

### SEQ IDS

Seq. Id. No. 1 shows the nucleotide sequence of human ILK.

Seq. Id. No. 2 shows the nucleotide sequence of human alpha-parvin.

Seq. Id. No. 3 shows the nucleotide sequence of human PINCH.

### FIGURES

FIG. 1: The changes of protein abundance with increasing compound concentration that was significantly changed by erlotinib hydrochloride, gefitinib or both are displayed. The changes are generally in good agreement with the expectations raised from the p-values (see FIG. 2). However, LIMS1 (PINCH) and M3K1 were only identified and thus quantified in erlotinib hydrochloride displaced samples, while EPHA1, K0564, KC1E, NUD12, SN1L1 were only found in gefitinib.

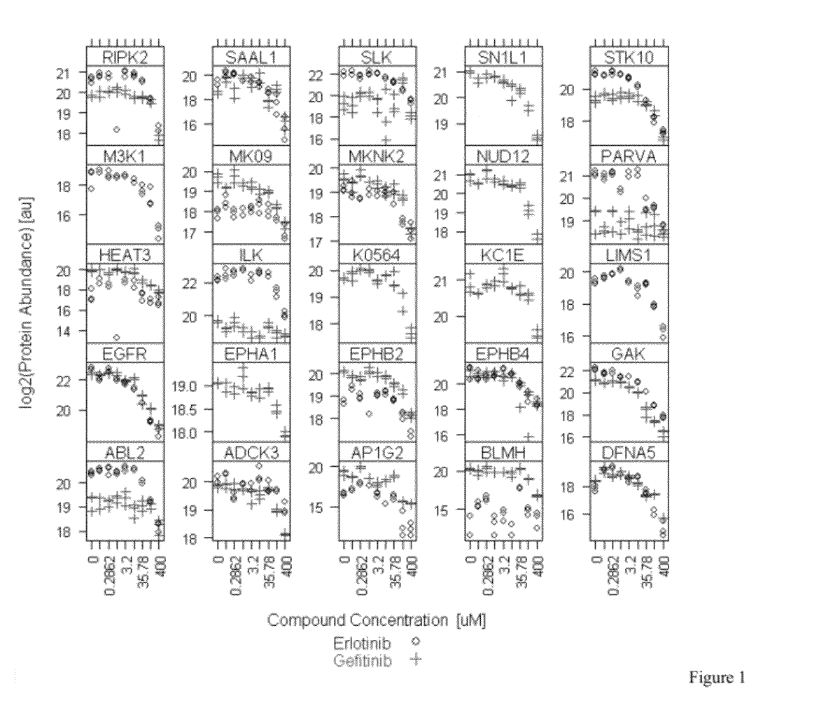

FIG. 2: The adjusted p-values of erlotinib hydrochloride vs. the adjusted p-values of gefitinib after log 10 transformation are shown (lines indicate 5% probability of error). Region A show proteins significantly binding to erlotinib hydrochloride and gefitinib, Region B and C show proteins significantly binding to erlotinib hydrochloride and gefitinib, respectively. Proteins in Region D did not show significant binding in neither of the two. This figure can only display proteins identified in both compounds, for this reason the proteins named above which are present in only erlotinib hydrochloride or gefitinib displaced samples are missing here.

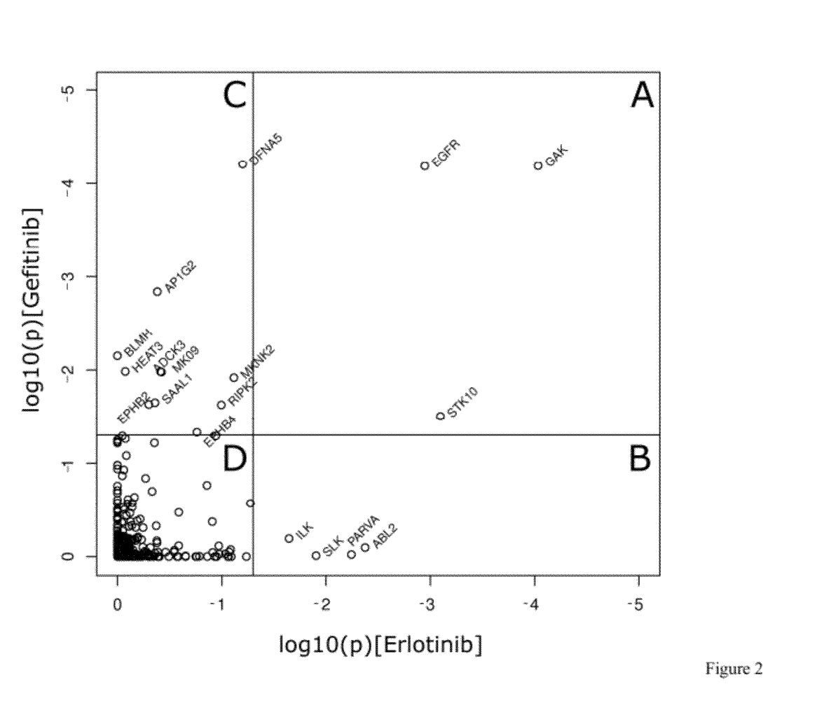

FIG. 3: Interaction profiles of erlotinib hydrochloride and gefitinib in various NSCLC cell lines. Whereas the relative amount of EGFR is similar in all cell lines, this figure shows the specific enrichment of ILK, alpha-parvin and PINCH in erlotinib hydrochloride bound fractions.

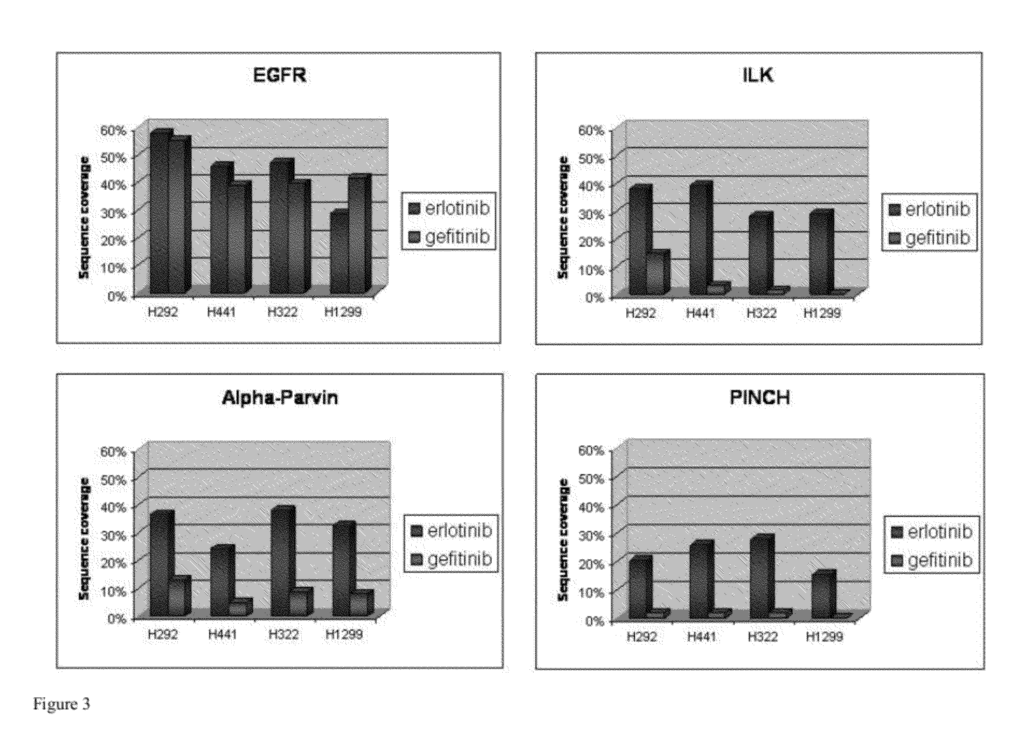

FIG. 4: ATP binding site of aligned and overlaid ILK and EGFR crystal protein structures showing potential interactions of erlotinib and gefitinib with the area of the ILK DFG-loop. Oxygen atoms are colored in red, nitrogen in blue, chlorine in green and fluorine in light cyan. Carbon atoms of the erlotinib/EGFR complex (1M17) are colored in salmon, carbon of the gefitinib/EGFR (2ITY) complex in yellow and carbon atoms of ILK are colored in white. Picture was generated with PyMOL. The different substitutions in the erlotinib and gefitinib aniline rings (the 3 position is substituted with chlorine in gefitinib but with acetylene in erlotinib and the 4 position is unsubstituted in erlotinib but substituted with fluorine in gefitinib). The resulting differences in atom proximities suggest that erlotinib is more likely to bind favorably to the ATP binding site of ILK compared to Gefitinib.

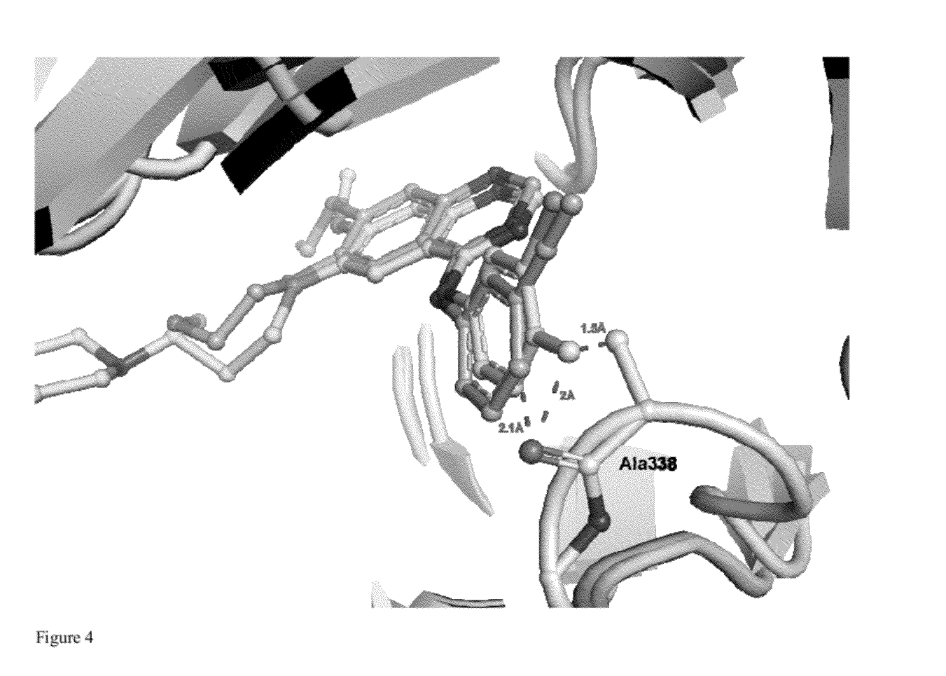

FIG. 5: Erlotinib slows down the transition of H358 by stabilizing E-cadherin. A) Western blot on E-cadherin. Quantified signals of B) E-cadherin, C) ILK and D) PINCH, normalized on total protein content and averaged between two separate experiments.

FIG. 6: IPP expression in NSCLC cell lines. A) Western blots of ILK, alpha-parvin, PINCH, E-cadherin, vimentin and GAPDH on EGFR wild-type NSCLC epithelial cell lines H292, H441, H358, H322, and mesenchymal cell lines H460, H1299, H226 and Calu6. B) Quantified Western blot signals normalized on GAPDH levels and averaged to determine the level of expression of the IPP complex.

FIG. 7: E-cadherin (CDH1) and ILK expression in patients with versus patients without clinical benefit. Each dot represents the gene signal (log 2 transformed) measured in one sample. Blue and red dots correspond to samples from patients with and without clinical benefit, respectively. (Dotplots performed with Partek software).

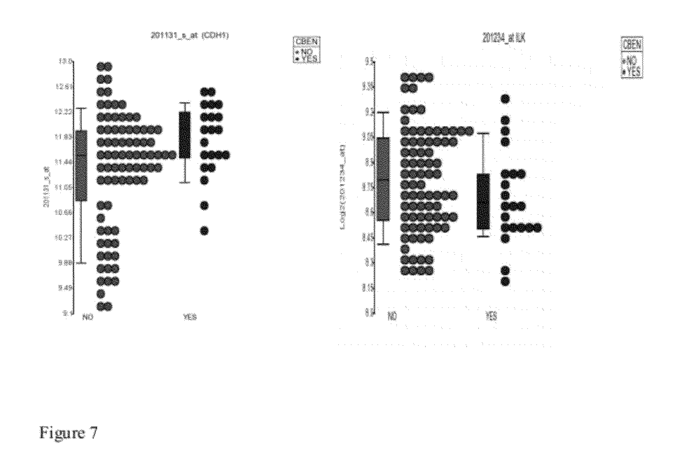

### EXAMPLE

The invention will now be further described in the example below, which is intended as an illustration only and does not limit the scope of the invention.

Cell Culture.

H292 cells were maintained in RPMI 1640 medium (Gibco-31870) supplemented with 10% FBS and penicillin/streptomycin/glutamine. A pellet of 1·10⁸ H292 cells was lysed in 2.5 mL buffer (150 mM NaCl (sodium chloride) 1 mM CaCl₂ (calcium chloride) MgCl₂ (magnesium dichloride), 1% NP40 (nonyl phenoxypolyethoxylethanol), complete EDTA (Ethylendiamin-tetraacetate) free protease inhibitors, 1 mM orthovanadate and 50 mM Hepes). After 10 minutes incubation on ice, the lysates were cleared by centrifugation for 15 minutes at 12,000×g at 4° C.

Compound Synthesis and Immobilization.

Erlotinib hydrochloride and gefitinib have been chemically modified to be covalently linked to epoxy-activated sepharose 6B beads (GE Healthcare). Coupling of the derivatives to epoxy-activated sepharose 6B was done in 50% dimethylformamide (DMF)/0.1 M sodium carbonate (Na₂CO₃) pH 11 in presence of 10 mM sodium hydroxide (NaOH) overnight at room temperature in the dark. After 3 washes with 50% dimethylformamide (DMF)/0.1 M sodium carbonate (Na₂CO₃), remaining reactive groups were blocked with 1 M ethanolamine. Subsequent washing steps were performed according to the manufacturer's instructions. Binding efficiency was estimated by comparing the optical densities of the compound solutions before and after coupling at the maximal absorbance wavelengths 247 and 330 nm.

Competition Experiment and Affinity Chromatography.

500 μg of the respective proteins were preincubated with increasing concentrations (0, 0.0856, 0.2862, 0.957, 3.2, 10.7, 35.78, 119.6 and 400 μM final concentration) of either erlotinib hydrochloride or gefitinib for 1 hour at 4° C. under rotation. Then, samples were mixed with equilibrated epoxy-activated Sepharose beads with immobilized erlotinib hydrochloride or gefitinib for 3 hours at 4° C. with rotation. Beads were then washed 3 times with lysis buffer and finally resuspended in 100 μL SDS loading buffer, boiled 5 minutes at 85° C. and centrifuged 5 minutes at 12,000×g.

SDS-PAGE.

Elution samples were separated on 4-20% Tris Glycine SDS-PAGE (Invitrogen, EC6025BOX). Gels were fixed 1 hour in 40% ethanol 10% acetic acid and stained overnight using Coomassie blue (Invitrogen LC6025).

Mass Spectrometry.

For in-gel digestion, an adapted protocol of Shevchenko et al. (1996) was used. Briefly, each lanesof the gel was cut into 9 slices and loaded onto a 96 wells Proxeon plate. Gel pieces were subsequently washed with milliQ water and acetonitrile, reduced in dithiothreitol and alkylated with iodoacetamide. After thorough washing, pieces were incubated with 50 ng trypsin (Promega, V5111) and allowed to digest overnight. Supernatants of the digests were collected, and peptides from the gel pieces extracted with one change of 25 mM ammonium bicarbonate followed by two changes of 5% formic acid. All supernatants were pooled, dried and resuspended in 2% acetonitrile, 5% formic acid and analyzed by LC-MS/MS using a nanoflow liquid chromatograph (Proxeon EASY_nLC) coupled to an electrospray ion trap mass spectrometer (Velos LTQ-Orbitrap, Thermo Scientific). Peptide separation was done on a ReproSil-PUR C18-AQUA 3 μM column coupled to an AQUA 100 microns trap column over a 60 minutes acetonitrile gradient.

Sequence Identification

Raw data files were processed using the SEQUEST search algorithm 27 (SEQUEST version 27.0, revision 12, Thermo Electron). Searches were performed against the SwissProt Human database (version 48, 219024 entries) with a mass tolerance of +/−10 ppm for parent ions and +/−1.0 Da for fragment ions. Methionines (reduced/oxidized; +15.9949 Da) were considered as differential modifications while cysteines were considered as fully carbamidomethylated (+58.0133 Da). Only fully tryptic peptides with no more than one miscleavage were considered for data analysis. Peptides were considered as unambiguously identified if their XCorr scores exceeded 2.0, 2.5 or 3.0 for singly, doubly and triply charged ions respectively and if the corresponding dCn scores were larger than 0.1. In this report, only proteins for which at least two different peptides were identified were further processed for quantification purposes.

Mass Spectrometric Data Processing by Genedata RefinerMS 5.2.6

For each compound and each band independently, the respective Thermo raw files were loaded by Genedata RefinerMS 5.2.6: Centroid data are discarded to reduce memory usage. Signals occuring in only one spectrum are removed; all data representations of samples (=chromatograms) are given a common 2D m/z-RT grid. Before Chromatogram Alignment which relies on appropriate peak finding parameters, peak finding parameters were visually inspected. Chromatogram Alignment corrects for retention time shifts between chromatograms, based on found common peaks and Trivial Tree Algorithm. The success of this operation was visually inspected. RT Range Restriction discards signals and data outside the information rich region of 23-52 min of chromatography. The Chromatogram Average activity computes a virtual average chromatogram of all input chromatograms. The level of noise is reduced by the “Intensity Threshold” workflow step, eliminating each signal below a threshold of 1000 au. Peaks and their boundaries in RT and m/z direction are found in, and visualized as peak-surrounding squares. The squares are projected on each chromatogram, and the eXtracted Ion Counts (XIC) are determined within these boundaries. The resulting spreadsheet with one row per peak and one column per chromatogram is saved as file in the next workflow step. The ScanID's of MS/MS scans found within these signal boundaries are saved in the same way. A report is generated that summarizes the workflow steps, their order parameters plus diagnostics information from the steps (e.g. degree of local chromatographic shift per sample, number of signals found).

Further Processing

For each compound and each band independently, the respective samples were normalized using the Quantile approach (Smyth 2003). The peaks found in RefinerMS are assigned with the sequence information via their ScanID in the respective measurement. Peaks without identification or with more ambiguous identification are removed. The quantitative information is log 2-transformed. For Process Control, quantitative data is subjected to Principal Component Analysis: Scores of the first 4 principal components were correlated with Compound Concentration and process parameters, such as gel lane number, LC-MS Batch ID or LC-MS run Number. Mahalanobis Distance outliers are estimated based on the scores of the first two principal components; samples outside the 95% confidence interval were considered as outliers and removed from further analysis.

Combination of Peptide intensities to Protein Surrogate

Peptides are assigned to their potential protein precursors. Each found combination of protein precursors is given a separate protein quantitation group, typically with only one protein member, or a protein and its splice variants. Following Irizarry et al. (2003), a relative abundance for each protein quantitation group is obtained from the normalized and log-transformed eXtracted Ion Counts (XIC), fitting a linear additive model:

XICᵢⱼₙ=μᵢₙαⱼₙ+εᵢⱼₙ, with αⱼ the peptide effect, μᵢ the log scale relative abundance of the protein quantitation group, and εᵢⱼ representing an independent identically distributed error term with mean 0. As described in Irizarry et al. (2003) and Holder et al. (2001), the median polish is employed to robustly estimate model parameters.

Detecting Significant Dose Response

A Hirotsu contras matrix is derived from the number compound concentrations (see Bretz et al. (2001)), the first and last contrast are eliminated. Moderated t-statistics T^{SC} are obtained for each contrast and protein quantitation group (see Smyth et al. (2004)). For each protein quantitation group, T^{MC}=max(T^{SC}) is obtained (see Bretz et al. (2001)). P-values are obtained based on the algorithm for step-down maxT adjusted p-values with 1000 permutations based on Ge et al. (2003), which originates from Westfall and Young (1993). It is modified with respect to the one-sidedness of the statistic by not choosing |T| but T.

Visualizations and any computations are performed with R 2.10.1 (citation “R”).

| | Signal Reduction (%) | |
|---|---|---|
| Protein | Gefitinib | Erlotinib hydrochloride |
| ILK | 0.0 | 77.3 |
| PINCH | 0.0 | 87.7 |
| alpha-parvin | 0.0 | 79.5 |

### Confirmation of Binding Profiles in Other NSCLC Cell Lines

H292, H441, H460 and H1299 cells were maintained in RPMI 1640 medium supplemented with 10% fetal bovine serum and 2 mmol/L L-glutamine. Compound synthesis, immobilization and affinity chromatography were performed as described above. Mass spectrometry analysis was performed on an LC system (LC-Packings) connected to a Thermo Finnigan LCQ Deca XP was used. Briefly, peptide extracts (in 5% acetonitrile, 0.5% acetic acid, 0.012% HFBA (heptafluorobutyric acid) with 1% formic acid) were injected on a trap column (PepMap™ C18, 5 μm (LC-Packings), 5 mm×300 μm I.D.) at 20 μL/min. Subsequently, the peptides were transferred in solvent A (5% acetonitrile, 0.5% acetic acid, 0.012% HFBA) on the analytical column (Magic C18™ 5 μm (Spectronex), 10 cm×75 μm I.D.). Elution was performed with a 3 step linear gradient from 5 to 15% solventB (0.5% acetic acid, 0.012% HFBA in 80% (v/v) acetonitrile) in 8 min, from 15 to 38% solvent B in 65 min and from 38 to 80% solvent B in 25 min. The column effluent was directly introduced into the ESI source of the mass spectrometer. The mass spectrometer was operated in the positive ion mode and parent ions were selected for fragmentation in data-dependent mode. Raw MS/MS data were searched against human SwissProt database using SEQUEST (version 27). Parameters included one missed cleavage allowed and a peptide mass tolerance of 1.5 Da.

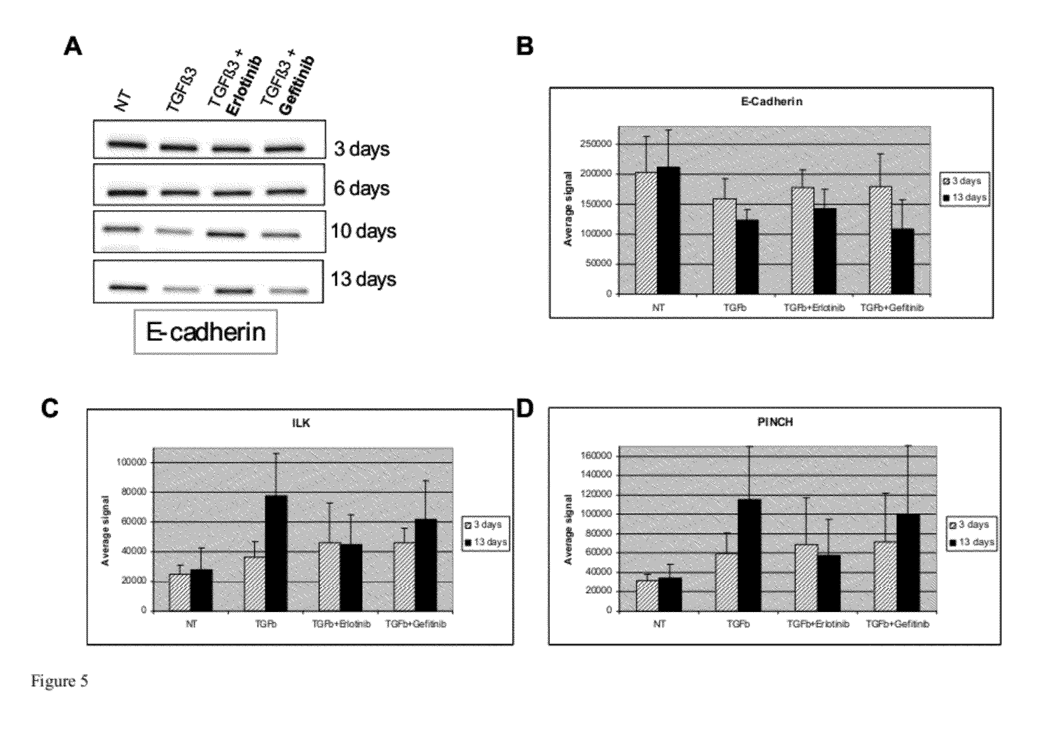

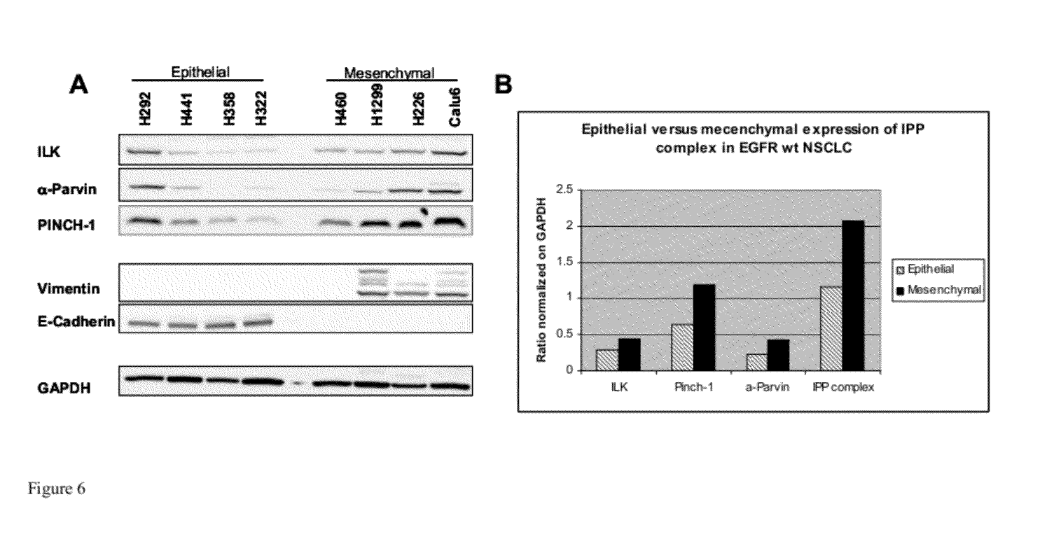

A model NSCLC cell line, H358, was used, which had been previously described as switching from epithelial to mesenchymal state upon TGFbeta3 treatment (Thomson 2008). The results show a decrease of E-cadherin after 13 days of TGFbeta3 treatment, indicating that the cells are actually transitioning (FIG. 5a). The level of E-cadherin in H358 cells treated with TGFbeta3 and erlotinib remained unchanged, unlike in cells treated with TGFbeta3 and gefitinib. The quantification of the western blot signals shows a decrease of more than ⅓ in E-cadherin levels (FIG. 6b) between non treated cells and cells treated for 13 days with TGFbeta3. The treatment with erlotinib prevents this decrease, while gefitinib fails to inhibit the TGFb3-mediated decrease of E-cadherin levels. (FIG. 5b). The effects on ILK and PINCH expression shows that the levels of both proteins increase after TGFb3 treatment (FIGS. 5c and d). The ILK and PINCH expression levels do not change when the cells are treated with erlotinib, an effect which was not so pronounced upon gefitinib treatment. This EMT model therefore, demonstrates that erlotinib reduces the extent of EMT by inhibiting the loss of E-cadherin and also suggests to prevent a TGFb3-mediated increase of ILK and PINCH.

The three components of the complex ILK, alpha-parvin and PINCH are studied on an individual basis, not addressing the possibility of the expression of the entire complex being correlated to an EMT status. Eight EGFR wild type NSCLC cell lines were selected depending on this status (Thomson 2005; Thomson 2008), which was assessed by the expression of E-cadherin as an epithelial marker and vimentin as a mesenchymal marker. All the IPP proteins were detected with various intensities in all eight cell lines except for alpha-parvin in H358 (FIG. 6a). After quantification and normalization, the overall sum of IPP complex intensities was 1.8 times higher in mesenchymal versus epithelial cells. PINCH1 was the most highly expressed protein (56% and 58% of total IPP expression in epithelial and mesenchymal cells respectively), followed by ILK (25% and 22%) alpha-parvin (19% and 20%) (FIG. 6b).

### E-cadherin and ILK Expression

The potential relationship between IPP and more global genes involved in EMT have been evaluated. Specifically the differences between their expression profiles in patients with and without clinical benefit to erlotinib. In total, only 16 out of the 21 pre-selected genes were identified with detected probe sets and were statistically analyzed. Among the 31 probe sets (corresponding to 16 unique genes), two were statistically significant differentially expressed in tumor samples from patients with and without clinical benefit. The two corresponding genes were ILK and CDH1 (E-cadherin), which are downregulated (0.93-fold, p=0.011) and upregulated (1.38-fold, p=0.033) respectively in patients with clinical benefit.

### Alignment of EGFR and ILK Crystal Protein Structures

Publicly available crystal structure pdb files of EGFR-bound erlotinib or gefitinib and the crystal structure of ILK complexed with adenosine triphosphate (ATP) were downloaded from the crystallographic database PDB (Berman 2000) (pdb codes 1M17, 2ITY, 3KMW; Stamos 2002; Yun 2007; Fukuda 2009). The three kinase structures were structurally aligned using the coordinates from Ca atoms of a subset of the residues forming the ATP binding site (residue numbers based on EGFR numbering 691-693, 701-703, 719-72 (19) 1, 751, 766-772, 820-823, 831-832. Alignment and distance measurements were done with the PyMOL visualization package.

### Testing EMT Transition on H358 NSCLC Cell Line

H358 cells were treated with recombinant 10 ng/mL TFGβ3 (Peprotech, New York, N.Y., USA) and erlotinib or gefitinib 5 μM for 3, 6, 10 and 13 days. Cell culture medium was renewed every 3 days. Cell lysis was performed in PBS containing 1% NP-40 and protease inhibitors. After clearing by centrifugation at 12,000 g for 15 min at 4° C., proteins were separated by SDS-PAGE and processed for western blot analysis. Images were acquired using a compact digital camera (Luminescent Image Analyzer: Fujifilm, Tokyo, Japan) and quantified with ImageQuant TL (GE Healthcare, Waukesha, Wis., USA).

### Analysis of MERIT Data

Gene expression profiles were measured in tumor biopsy samples from 102 patients with stage IIIb/IV NSCLC, being treated with erlotinib (150 mg/day). Tumor biopsies were analyzed using gene expression profiling with Affymetrix GeneChip Human Genome U133A 2.0 Array (Affymetrix, Santa Clara, Calif., USA). All gene expression results described here (fold changes and p values) come from the statistical analysis already reported (Tan 2010). We specifically looked at differences between gene expression profiles of patients with and without clinical benefit from erlotinib. The following 21 proteins, potentially involved in transcriptional regulation of EMT or in IPP complex, were mapped to the corresponding genes and microarray probe sets: Twist, Snail [Sma1], Zeb [TCF8], Lef-1, E12/E47 basic Loop helix [bHLH], Slug [Sma2], E-cadherin [CDH1], N-cadherin [CDH2], Myc, Vimentin, Catenin beta, Catenin delta, Fibronectin, Epimorphin, integrin-linked kinase [ILK], Parvin alpha, Parvin beta, PINCH 1, PINCH 2, Metastasis associated protein 3 [MTA3], Y box binding protein 1 [YB-1]).

### REFERENCES

Shevchenko, A.; Wilm, M.; Vorm, O.; Mann, M. Mass spectrometric sequencing of proteins silver-stained polyacrylamide gels. Anal Chem 1996; 68, 850-58.

Smyth, G. K., and Speed, T. P. (2003). Normalization of cDNA microarray data. Methods 31, p 265-273.

Smyth, G. K., Michaud, J., and Scott, H. (2005). The use of within-array replicate spots for assessing dierential expression in microarray experiments. Bioinformatics 21(9), 2067-2075.

Irizarry R A, Hobbs B, Collin F, Beazer-Barclay Y D, Antonellis K J, Scherf U, Speed T P. (2003) Exploration, normalization, and summaries of high density oligonucleotide array probe level data. Biostatistics Apr; 4(2):249-64.

Holder, D, Raubertas, R F, Pikounis, V B, Svetnik, V and Soper, K (2001). Statistical analysis of high density oligonucleotide arrays: a SAFER approach. Proceedings of the ASA Annual Meeting 2001. Atlanta, Ga.

Bretz F, Hothorn L A. (2001) Testing dose-response relationships with a priori unknown, possibly nonmonotone shapes. J Biopharm Stat. 11(3):193-207.

Smyth G K. (2004) Linear models and empirical bayes methods for assessing differential expression in microarray experiments. Stat Appl Genet Mol Biol.; 3:Article3. Epub 2004 Feb. 12.

Bretz F, Hothorn L A. (2002) Detecting dose-response using contrasts: asymptotic power and sample size determination for binomial data. Stat Med. 2002 21(22):3325-35.

P. H. Westfall and S. S. Young (1993). Resampling-based multiple testing: Examples and methods for p-value adjustment. John Wiley & Sons, Algorithm 4.1, p 116-117.

R Development Core Team (2009). R: A language and environment for statistical computing. R Foundation for Statistical Computing, Vienna, Austria. ISBN 3-900051-07-0, URL http://www.R-project.org.

Ge, Y. et al. (2003) Resampling-based multiple testing for microarray data analysis, Test, 12(1), 1-77.

Thomson et al.; 2005; Cancer Res.; 65; 9455-9462.

Takanami, I.; 2005; BMC Cancer; 5; 1-7.

Berman, H. M., Westbrook, J., Feng, Z., Gilliland, G., Bhat, T. N., Weissig, H., Shindyalov, I. N., and Bourne, P. E. (2000) The Protein Data Bank. pp. 235-242.

Stamos, J., Sliwkowski, M. X., and Eigenbrot, C. (2002) Structure of the epidermal growth factor receptor kinase domain alone and in complex with a 4-anilinoquinazoline inhibitor. J Biol Chem 277, 46265-46272.

Yun, C. H., Boggon, T. J., Li, Y., Woo, M. S., Greulich, H., Meyerson, M., and Eck, M. J. (2007) Structures of lung cancer-derived EGFR mutants and inhibitor complexes: mechanism of activation and insights into differential inhibitor sensitivity. Cancer Cell 11, 217-227.

Fukuda, K., Gupta, S., Chen, K., Wu, C., and Qin, J. (2009) The pseudoactive site of ILK is essential for its binding to alpha-Parvin and localization to focal adhesions. Mol Cell 36, 819-830.

Tan, E. H., Ramlau, R., Pluzanska, A., Kuo, H. P., Reck, M., Milanowski, J., Au, J. S., Felip, E., Yang, P. C., Damyanov, D., Orlov, S., Akimov, M., Delmar, P., Essioux, L., Hillenbach, C., Klughammer, B., McLoughlin, P., and Baselga, J. (2010) A multicentre phase II gene expression profiling study of putative relationships between tumour biomarkers and clinical response with erlotinib in non-small-cell lung cancer. Ann Oncol 21, 217-222.

Yauch, R. L., Januario, T., Eberhard, D. A., Cavet, G., Zhu, W., Fu, L., Pham, T. Q., Soriano, R., Stinson, J., Seshagiri, S., Modrusan, Z., Lin, C. Y., O'Neill, V., and Amler, L. C. (2005) Epithelial versus mesenchymal phenotype determines in vitro sensitivity and predicts clinical activity of erlotinib in lung cancer patients. Clin Cancer Res 11, 8686-8698.

Cabodi, S., Del Pilar Camacho-Leal, M., Di Stefano, P., and Defilippi, P. (2010) Integrin signalling adaptors: not only figurants in the cancer story. Nat Rev Cancer 10, 858-870.

Thomson, S., Buck, E., Petti, F., Griffin, G., Brown, E., Ramnarine, N., Iwata, K. K., Gibson, N., and Haley, J. D. (2005) Epithelial to mesenchymal transition is a determinant of sensitivity of non-small-cell lung carcinoma cell lines and xenografts to epidermal growth factor receptor inhibition. Cancer Res 65, 9455-9462.

Thomson, S., Petti, F., Sujka-Kwok, I., Epstein, D., and Haley, J. D. (2008) Kinase switching in mesenchymal-like non-small cell lung cancer lines contributes to EGFR inhibitor resistance through pathway redundancy. Clin Exp Metastasis 25, 843-854.

Singh, A., and Settleman, J. (2010) EMT, cancer stem cells and drug resistance: an emerging axis of evil in the war on cancer. Oncogene 29, 4741-4751.

Oloumi, A., McPhee, T., and Dedhar, S. (2004) Regulation of E-cadherin expression and beta-catenin/Tcf transcriptional activity by the integrin-linked kinase. Biochim Biophys Acta 1691, 1-15.

1. An in vitro method of predicting the response of a cancer patient to treatment with erlotinib hydrochloride comprising:
determining the expression levels of one, two or three genes selected from the group consisting of ILK, alpha-parvin and PINCH genes in a tumor sample of a patient and comparing the expression levels of the ILK, alpha-parvin and PINCH genes together or individually to a value representative of the expression levels of ILK, alpha-parvin and PINCH genes together or individually in tumors of a population of patients deriving no clinical benefit from the treatment, wherein a lower expression level of ILK, alpha-parvin and PINCH genes together or individually in the tumor sample of the patient is indicative for a patient who will derive clinical benefit from the treatment.

2. The method according to claim 1, wherein the expression level is determined by microarray technology.

3. An in vitro method of predicting the response of a cancer patient to treatment with erlotinib hydrochloride comprising:
determining the activation level of one, two or three proteins selected from the group consisting of ILK, alpha-parvin and PINCH proteins, in a tumor sample of a patient and comparing the activation levels of the ILK, alpha-parvin and PINCH proteins together or individually to a value representative of the activation levels of ILK, alpha-parvin and PINCH proteins together or individually in tumors of a population of patients deriving no clinical benefit from the treatment, wherein a lower activation level of ILK, alpha-parvin and PINCH proteins together or individually in the tumor sample of the patient is indicative for a patient who will derive clinical benefit from the treatment.

4. An in vitro method of predicting the response of a cancer patient to treatment with erlotinib hydrochloride comprising:
determining the phosphorylation levels of ILK protein in a tumor sample of a patient and comparing the phosphorylation levels of the ILK protein to a value representative of the phosphorylation levels of ILK protein in tumors of a population of patients deriving no clinical benefit from the treatment, wherein a different phosphorylation level of ILK protein, in the tumor sample of the patient is indicative for a patient who will derive clinical benefit from the treatment.

5. The method according to claim 1, wherein the expression levels of each of ILK, alpha-parvin and PINCH are determined in such tumor sample.

6. The method according to claim 1, wherein the expression level of the ILK gene is determined in such tumor sample.

7. The method according to claim 1, wherein the expression level of the alpha-parvin gene is determined in such tumor sample.

8. The method according to claim 1, wherein the expression level of the PINCH gene is determined in such tumor sample.

9. The method according to claim 3, wherein the expression levels of each of ILK, alpha-parvin and PINCH genes are determined in such tumor sample.

10. The method according to claim 3, wherein the expression level of the ILK gene is determined in such tumor sample.

11. The method according to claim 3, wherein the expression level of the alpha-parvin gene is determined in such tumor sample.

12. The method according to claim 3, wherein the expression level of the PINCH gene is determined in such tumor sample.

13. The method according to claim 1, wherein the cancer is NSCLC.

14. The method according to claim 1, wherein a cancer patient does not respond to treatment with gefitinib.

15. Use of one, two or three genes selected from the group consisting of ILK, alpha-parvin and PINCH genes for predicting the response of a cancer patient to erlotinib hydrochloride treatment.

16. Use of erlotinib hydrochloride for treating cancer, wherein
i) the activation levels of one, two or three proteins selected from the group consisting of ILK, alpha-parvin and PINCH proteins in a tumor sample of a patient are measured,
ii) the expression levels of the ILK, alpha-parvin and PINCH genes together or individually are compared to a value representative of the expression levels of ILK, alpha-parvin and PINCH genes together or individually in tumors of a population of patients deriving no clinical benefit from the treatment, and
iii) erlotinib hydrochloride is administered to a patient having a lower expression level of ILK, alpha-parvin and PINCH genes together or individually.

17. Use according to claim 16, wherein the activation level of ILK protein is measured and the expression level of the ILK gene is compared.

18. Use according to claim 16, wherein the activation level of alpha-parvin protein is measured and the expression level of the alpha-parvin gene is compared.

19. Use according to claim 16, wherein the activation level of PINCH protein is measured and the expression level of the PINCH gene is compared.

20. The use according to claim 16, wherein the cancer is NSCLC.
